(19) United States
(12) Patent Application Publication
(10) Pub. No.: US 2023/0082205 A1
(43) Pub. Date: Mar. 16, 2023

(54) DECOUPLING DELIVERY SYSTEM

(71) Applicants: Mustafa Hasan Mohamad Abdullah AlQbandi, Kuwait City (KW); Eran Levit, Amherst, NH (US)

(72) Inventors: Mustafa Hasan Mohamad Abdullah AlQbandi, Kuwait City (KW); Eran Levit, Amherst, NH (US)

(21) Appl. No.: 17/795,214

(22) PCT Filed: Jan. 25, 2021
(86) PCT No.: PCT/US21/14954

§ 371 Date: Jul. 25, 2022

Related U.S. Application Data
(60) Provisional application No. 62/965,000, filed on Jan. 23, 2020.

(51) Int. Cl. A61M 25/00

(52) U.S. Cl. CPC A61M 25/00; A61M 25/01

(57) ABSTRACT

A decoupling delivery system may include a handle portion and a catheter portion. The handle portion may be removable coupled to the catheter potion. The catheter portion may include a cable extending from the handle portion to a distal end of the catheter portion and having an inner lumen. The system may include a wire extending through the inner lumen and the wire may be operable by actuation of the handle to advance or retract the wire within the inner lumen. The system may have a device engagement mechanism arranged at the distal end configured for engaging a medical device in response to longitudinal motion of the wire.

### CROSS REFERENCE TO RELATED APPLICATION

The present application claims the benefit of U.S. Provisional Application No. 62/965,000, filed on 23 Jan. 2020, the entire contents of which are incorporated herein by reference. A claim of priority to all, to the extent appropriate, is made.

### TECHNOLOGICAL FIELD

The present disclosure relates to a system and method for in situ disconnection and reconnection and/or splicing of a medical device delivery cable. More particularly, the present disclosure relates to a system and method for separating a handle portion from a catheter portion of a delivery system and later reconnecting them. In one or more embodiments, a cable running through the delivery system may be supplemented with an additional piece of cable while the handle portion is disconnected. Still more particularly, the system and method of disconnection and reconnection may include a mechanism providing for constant secured control over a medical device connected to a distal end of the delivery cable during disconnection and reconnection.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Several medical procedures involve delivery of a medical device through a delivery sheath. In these instances, the medical device being delivered is positioned on a leading end of a cable or wire. The medical device is then advanced through the delivery sheath using the cable or wire and may be deployed by advancing the device out the distal end of the delivery sheath. Several problems exist in this context.

First, as may be appreciated, the cable or wire used to advance and deploy the medical device may have a length sufficient to extend from an entry point into the sheath to the location within the body where the device is to be deployed. However, in some circumstances, the cable or wire provided may have insufficient length to reach the deployment location. Second, sometimes the medical device that is being deployed or placed may expand, reform, or otherwise change shape during deployment and the placement of the medical device may not go as planned such that removal and replacement of the device may be desired. However, given that the medical device may have changed shape during placement, withdrawing the device into the or sheath may create difficulties. This is particularly true where the device cannot be readily reshaped into its pre-deployment or delivery shape. In these circumstances, it may be useful to be able to remove the sheath and replace it with a larger one to allow for withdrawal of the device into the larger sheath. Third, the sheath may be damaged preventing the removal/replacement of the medical device. Fourth, the sheath may be pre-shaped to meet particular anatomies. Sometimes the pre-shaped sheath may not meet the anatomy as intended and may not provide for placement of the medical device. In this circumstance, exchange of the sheath may be a solution and maintaining the medical device inside the target treatment area during replacement of the sheath may be preferred rather than removing the sheath with the medical device and risk damaging the vasculature or wasting valuable time reaching the target the site.

Particular systems for delivering medical devices may not allow extending the length of the cable or wire to address the first problem mentioned above or for removal of the sheath to address the second, third, and fourth problems. For example, some systems for delivering the medical device may include a handle on a proximal end of the cable or wire. That is, the system used to deliver the medical device may include a cable or wire with a handle at a proximal end and an engagement mechanism on a distal end. The handle may be operably coupled to the engagement mechanism on the distal end of the cable/wire to allow the handle to operate the engagement system so that it may grasp, hold, and release the medical device. More particularly, the system may be equipped with a medical device by securing the medical device to the engagement mechanism. The handle may be manipulated to grasp the medical device and hold the medical device securely as it is advanced to its deployment location. After advancing the medical device to its proper location, the handle may be used to place and release the medical device. One example of such a device is the Pistol Pusher manufactured and sold by Occlutech. Another example of such a device is the Flex-II Pusher also manufactured and sold by Occlutech. These particular devices are permanently attached to the cable or wire that is used to advance and deploy the medical device and the cable or wire has a defined length. The permanent attachment relates to the concern that continuous control of the cable or wire may be important to maintaining constant secured control over the medical device attached at the distal end of the system such that the medical device is controlled unless and until it is placed in its proper location.

With respect to first problem identified above where the cable/wire has an insufficient length, the present systems are not usable because they cannot reach the deployment location so a longer system may need to be used. For the second, third, and fourth problems where the sheath is to be removed/replaced with a larger one or removed/replaced due to damage or incompatible pre-shaping, the permanent attachment of the handle on the system prevents removal and replacement of the sheath.

### SUMMARY

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

A decoupling delivery system is provided. In one or more embodiments, the decoupling delivery system includes a handle portion and a catheter portion, wherein the handle portion may be decoupled from the catheter portion while maintaining control of a medical device arranged on a distal end of the catheter. The handle may be operable by a user to deliver a medical device. The catheter portion may include a cable extending distally from the handle portion and having an engagement mechanism arranged at the distal end for engaging the medical device for delivery.

The handle may have a proximal portion, a core, distal portion, and a catheter engagement mechanism at the distal portion. A biasing element may be provided over the core separating the distal portion from the proximal portion.

The catheter portion may have a proximal end and a distal end. A handle engagement mechanism may be provided at the proximal end and a device engagement mechanism may be provided at the distal end. A cable having an inner lumen may extend from the proximal end to the distal end. A wire may extend through the inner lumen from the proximal end to the distal end and may be operable by actuation of the handle to advance or retract the wire longitudinally within the inner lumen. The device engagement mechanism may be operable to engage and disengage the medical device in response to longitudinal motion of the wire.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1a is a front view of a delivery system in place in a patient and delivering a medical device, according to one or more embodiments.

FIG. 1b is a schematic view of a delivery system for delivering a medical device, according to one or more embodiments.

FIG. 2 is a schematic side view showing a control wire within an outer cable for controlling an engagement mechanism at a distal end, according to one or more embodiments.

FIG. 3 is a schematic side view of the control wire and cable of FIG. 2 with the wire in a retracted position and the engagement mechanism in a closed condition, according to one or more embodiments.

FIG. 4 is a perspective view of an example engagement mechanism such as the one shown schematically in FIGS. 2 and 3 and arranged adjacent a medical device that the system may be used to deliver, according to one or more embodiments.

FIG. 5 is a perspective view of an engagement mechanism, according to one or more embodiments.

FIG. 6 is a front view thereof with the prongs of the engagement mechanism in an open or receiving condition, according to one or more embodiments.

FIG. 7 is a front view thereof with the prongs of the engagement mechanism in a closed or clasped condition, according to one or more embodiments.

FIG. 8a is a perspective view of a handle portion in a locked position, according to one or more embodiments.

FIG. 8b is a perspective view of a handle portion in an unlocked position, according to one or more embodiments.

FIG. 9 is a closer view of a catheter engagement mechanism, according to one or more embodiments.

FIG. 10 is a closer view of a catheter engagement mechanism, according to one or more embodiments.

FIG. 11 is a perspective view of a proximal portion of a catheter portion, according to one or more embodiments.

FIG. 12 is a schematic view of a proximal catheter portion of a catheter portion and a catheter engagement mechanism of a handle portion pre-engagement, according to one or more embodiments.

FIG. 13a illustrates engagement of a catheter engagement mechanism of a handle portion with a handle engagement mechanism of a catheter portion prior to deployment of a medical device, according to one or more embodiments.

FIG. 13b illustrates engagement of a catheter engagement mechanism of a handle portion with a handle engagement mechanism of a catheter portion during deployment of a medical device, according to one or more embodiments.

FIG. 14 is a cross-sectional view of a handle portion of a delivery device, according to one or more embodiments.

FIG. 15a is a cross sectional view of a handle portion in a locked position, according to one or more embodiments.

FIG. 15b is a cross sectional view of a handle portion in an unlocked position, according to one or more embodiments.

FIG. 15c is a cross sectional view of a handle portion in an unlocked position with the catheter distal end opened, according to one or more embodiments.

FIG. 16a is a perspective view of a handle portion and a proximal portion of a delivery device with the catheter portion and the handle portion coupled and the handle portion in a locked position, according to one or more embodiments.

FIG. 16b is a cross sectional view of a proximal catheter portion of a catheter portion and a catheter engagement mechanism of a handle portion coupled and the handle in a locked position, according to one or more embodiments.

FIG. 16c a perspective view of distal end of a catheter portion of a delivery device and a medical device for delivery, when a handle portion of the delivery device is in an unlocked position, according to one or more embodiments.

FIG. 17a is a perspective view of a handle portion and proximal portion of a catheter portion of a delivery device with the handle portion in an unlocked position, according to one or more embodiments.

FIG. 17b is a perspective view of distal end of a catheter portion of a delivery device and a medical device for delivery, when a handle portion of the delivery device is in an unlocked position, according to one or more embodiments.

FIG. 18a is a perspective view of a handle portion and a proximal portion of a delivery device with the handle portion in a locked position during decoupling of the handle portion from the catheter portion, according to one or more embodiments.

FIG. 18b is a cross sectional view of a proximal catheter portion of a catheter portion and a catheter engagement mechanism of a handle portion during decoupling with the handle in a locked position, according to one or more embodiments.

FIG. 19a illustrates a cross sectional view of cable to cable operation, according to one or more embodiments.

FIG. 19b illustrates an alternative view of cable to cable operation, according to one or more embodiments.

FIG. 20 is a cross-sectional view of a handle portion of another delivery system, according to one or more embodiments.

FIG. 21 is a close-up view of part of the delivery system of FIG. 20.

FIG. 22 is a lateral cross-sectional view of the delivery system of FIG. 20.

FIG. 23 is a longitudinal cross-sectional view of the delivery system of FIG. 20, according to one or more embodiments.

FIG. 24A is a perspective view of a coupling element of the delivery system of FIG. 10, according to one or more embodiments.

FIG. 24B is a perspective view of an insert for the coupling element of FIG. 24A, according to one or more embodiments.

FIG. 25 is a longitudinal cross-sectional view of the delivery system of FIG. 20 with the locking mechanism in a locked or proximal position and including three lateral cross-sectional views along the length thereof, according to one or more embodiments.

FIG. 26 is a longitudinal cross-sectional view of the delivery system of FIG. 20 with the locking mechanism in an unlocked or distal position, according to one or more embodiments.

FIG. 27 is a longitudinal cross-sectional view of the delivery system of FIG. 20 with the locking mechanism in a locked or proximal position and including a lateral cross-sectional view along a length thereof, according to one or more embodiments.

FIG. 28 is a longitudinal cross-sectional view of the delivery system of FIG. 20 with the locking mechanism in a locked or proximal position, according to one or more embodiments.

FIG. 29 is an exploded cross-sectional view of the delivery system of FIG. 20 showing the cable and wire decoupled from the handle, according to one or more embodiments.

FIG. 30 is a longitudinal cross-sectional view of the handle of the delivery system of FIG. 20 with the locking mechanism in an unlocked or distal position, according to one or more embodiments.

FIG. 31 is a longitudinal cross-sectional view of the handle of the delivery system of FIG. 20 with the locking mechanism in a locked or proximal position, according to one or more embodiments.

FIG. 32 is a close-up cross-sectional view of a distal area of the handle of the system of FIG. 20, according to one or more embodiments.

FIG. 33 is a perspective view of the concentric elements used for decoupling of the cable/wire from the handle and showing the distal guide of the coupling element adjacent a keyway on the cable tying element, according to one or more embodiments.

FIG. 34 is a perspective view like FIG. 33, but with the distal guide of the coupling element engaged in the keyway of the cable tying element, according to one or more embodiments.

FIG. 35 is a perspective view like FIG. 33, with the proximal guide of the cable tying element engaged in a keyway of the core element, according to one or more embodiments.

FIG. 36 is a perspective view like FIG. 35, with the proximal guide of the cable tying element adjacent a keyway of the core element, according to one or more embodiments.

FIG. 37 is a perspective view of an opposite side of the concentric elements used for decoupling and showing the proximal guide of the cable tying element engaged in a keyway of the core element and misalignment of the slots and windows in the concentric elements.

FIG. 38 is a perspective view like FIG. 37, but with the proximal guide adjacent the keyway in the core element and showing alignment of the slots and windows in the concentric elements.

FIG. 39 is a perspective view like FIG. 38, but showing the proximal portion of the wire sprung from its position within the concentric elements.

FIG. 40 is a perspective view like FIGS. 37-39, but with the release element and the proximal portion of the wire moved away from the core element, the coupling element, and the cable tying element.

FIG. 41 is a perspective view like FIGS. 37-40 showing the progressive decoupling of the coupling element, cable tying element, and distal portion of the cable/wire from the core element.

FIG. 42 is a perspective view of the handle of FIG. 20 showing the release window in the core element for the proximal portion of the wire, according to one or more embodiments.

FIG. 43A is a side view of an extension element poised for positioning on the portion of the delivery system remaining in a patient, according to one or more embodiments.

FIG. 43B is a side view of an extension element beginning initial engagement on the portion of the delivery system remaining in a patient, according to one or more embodiments.

FIG. 43C is a side view of an extension element continuing with engagement of the portion of the delivery system remaining in a patient, according to one or more embodiments.

FIG. 43D is a side view of an extension element fully advanced onto the portion of the delivery system remaining in a patient, according to one or more embodiments.

FIG. 43E is a side view of an extension element being rotated relative to the portion of the delivery system remaining in a patient, according to one or more embodiments.

FIG. 43F is a side view of an extension element being rotated further relative to the portion of the delivery system remaining in a patient, according to one or more embodiments.

FIG. 43G is a side view of an extension element having a biasing element being compressed, according to one or more embodiments.

FIG. 43H is a side view of an extension element being rotated to set the biasing element, according to one or more embodiments.

FIG. 44 is a diagram of a series of method steps for deploying a medical device and utilizing the decoupling features of the delivery system describe herein, according to one or more embodiments.

### DETAILED DESCRIPTION

The present disclosure, in one or more embodiments, relates to a decoupling delivery system wherein a handle or handle portion at a proximal end of the system may be removed while maintaining secured control over a medical device arranged at a distal end of the system. Removal of the handle may allow removal and replacement of a delivery sheath through which the delivery system was inserted and while the system and medical device remain in a patient. Upon replacement of the sheath, the proximal portion of the delivery system may be reconnected and used to continue deployment of the medical device, to retrieve the medical device, and/or replace the medical device. In other situations, for example, if a delivery system has an insufficient length to reach a deployment location within a patient, the delivery system may be extended by disconnecting the proximal portion, attaching an extension, and reattaching the proximal portion. In both cases, the system may maintain secured control over the medical device during disconnection and reconnection of the proximal portion. Still other details and approaches to use of the system are included in the following detailed description.

In general, the decoupling delivery system may include a handle portion and a catheter portion. The handle portion may be removable coupled to the catheter potion. The catheter portion may include a sheath/tube extending from the handle portion to a distal end of the catheter portion and having an inner lumen. The system may include a wire extending through the inner lumen and the wire may be operable by actuation of the handle to advance or retract the wire within the inner lumen. The system may have a device engagement mechanism arranged at the distal end configured for engaging a medical device in response to longitudinal motion of the wire.

It is to be appreciated that the general teachings of a catheter engagement mechanism of a handle portion and a handle engagement mechanism of a catheter portion may be used to retrofit existing delivery devices to enable a handle of the delivery device to be decoupled from a catheter portion of the delivery device, while maintaining control of a medical device engaged by the catheter portion of the delivery device.

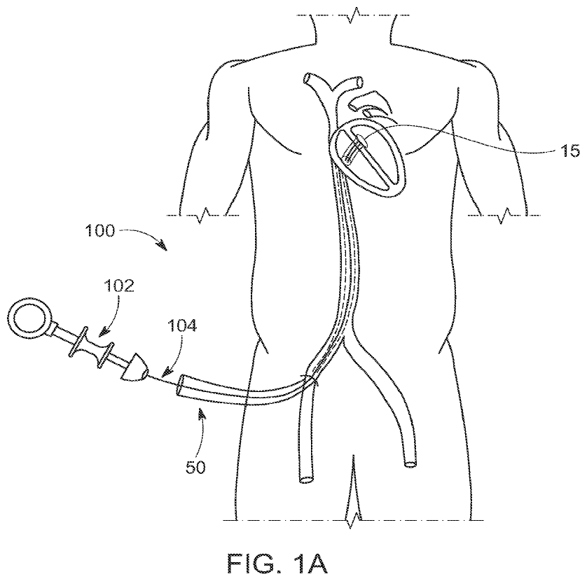

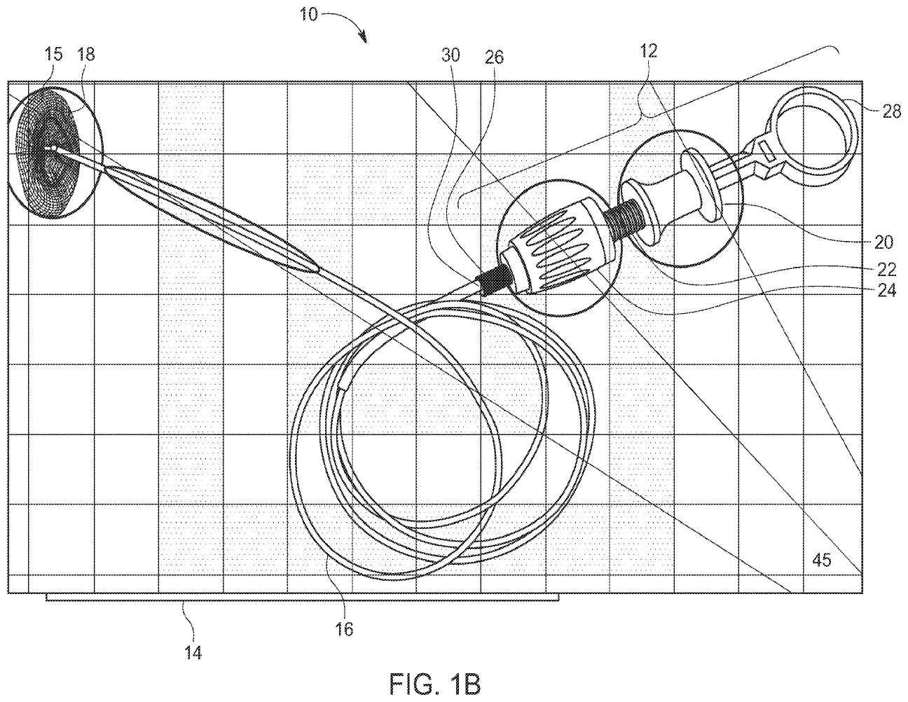

FIG. 1a illustrates a front view of a delivery system 100 is shown in place in a patient and delivering a medical device 15. The delivery system 100, in this example, is shown being used to deliver the medical device 15 to the heart of a patient. FIG. 1 illustrates the delivery system 100, including an operable handle portion 12, a device engagement mechanism 106, and a wire/cable 104 extending there between. All or part of the delivery system 100 may be provided within a sheath 50. For example, the wire/cable 104 may extend within a sheath 50. The medical device 15 may be positioned on the wire/cable 104 in order to access to a heart location such as a defect location, for example. Wire/cable refers to a cable having a wire extending there through. Such cable may alternatively be referred to as a sheath or tube.

FIG. 1b illustrates a perspective view of a decoupling delivery system 10. As shown, the decoupling delivery system 10 may include a handle portion 12 and a catheter portion 14, wherein the handle portion 12 may be decoupled from the catheter portion 14 while maintaining control of a medical device 15 arranged on a distal end of the catheter. The handle may be operable by a user to deliver the medical device. The catheter portion 14 may include a cable 16 extending distally from the handle portion 12 and having a device engagement mechanism 18 arranged at the distal end for engaging the medical device 15 for delivery.

The handle may have a proximal portion 20, a core 22, distal portion 24, a catheter engagement mechanism 26 and a thumb ring 28. A biasing element may be provided over the core 22 for separating the distal portion 24 from the proximal portion 20. The proximal portion 20 may also be referred to herein as an engagement control. The distal portion 24 may also be referred to herein as a locking mechanism.

The catheter portion may have a proximal end and a distal end. A handle engagement mechanism 30 may be provided at the proximal end and a device engagement mechanism 18 may be provided at the distal end. A cable 16 having an inner lumen may extend from the proximal end to the distal end. A wire may extend through the inner lumen from the proximal end to the distal end and may be operable by actuation of the handle to advance or retract the wire longitudinally within the inner lumen. The device engagement mechanism 18 may be operable to engage and disengage the medical device 15 in response to longitudinal motion of the wire.

The catheter engagement mechanism 26 of the handle portion 12 may work with the handle engagement mechanism 30 of the catheter portion 14 to releasably couple the handle portion 12 and the catheter portion 14 while allowing control of the medical device 15 even with the handle portion 12 decoupled from the catheter portion 14.

The handle portion 12 may be used to actuate the device engagement mechanism 18 via the cable/wire extending between the two. More particularly, the handle portion 12 may be used to release and/or grab a portion of the medical device 15. The delivery system 10 may be used to advance the medical device 15 through a sheath to a delivery location. The delivery system may advance the medical device out of a distal end of the sheath and may place the device in a suitable position on or in the patient. Once the device is properly placed, the handle portion 12 may be operated to release the grip of the device engagement mechanism 18 on the medical device 15 and the delivery system 10 may be withdrawn. As will be discussed in more detail below, in some situations, removal of the device, repositioning of the device, or other procedures may be performed, which may involve removal of the sheath while the delivery system and the delivered device remain in place in the patient. Given the size of the handle portion 12, the sheath may not be able to be removed with the handle in place and a splice may be provided to allow for removal of the handle. As may be appreciated, maintaining a substantially constant and secure grasp on the medical device 15 during removal and replacement of the handle may be important to avoid inadvertent release of the medical device within the patient.

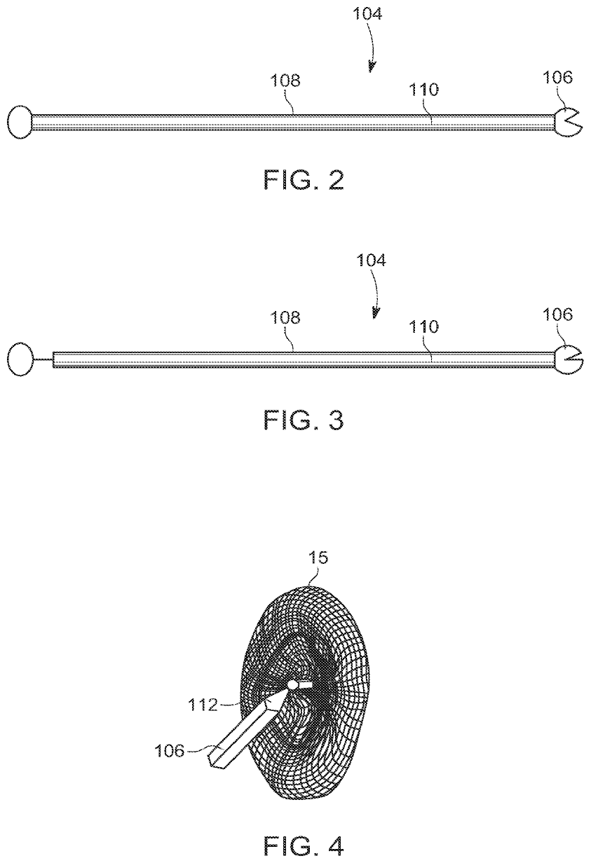

FIGS. 2 and 3 illustrate a schematic side view showing a wire 104 within a cable for controlling a device engagement mechanism 106 provided at a distal end of the wire. The cable may also be referred to herein as a sheath or tube. The cable/wire may extend to a distal end where an engagement mechanism 106 may be arranged. As shown schematically in FIGS. 2 and 3, the cable/wire system 104 extending between the handle portion and the device engagement mechanism 106 may be a two-part element including a cable 108 and a wire 110 arranged within the cable. The cable 108 may include a braided metal mesh element, a coiled wire, or another formation of a cable. The cable 108 may include a lumen extending throughout its length to accommodate the wire 110. The wire 110 may be arranged within the cable 108. The cable 108 and the wire 110 may be connected to the handle portion 12 at a proximal end and may extend to the distal end. At the distal end, the internal wire 110 may be connected to a device engagement mechanism 106. The handle portion 12 may be used to actuate the device engagement mechanism 106 to secure the medical device at the distal end. As shown in FIG. 3, the operation of the handle portion 12 may draw the wire 110 proximally relative to the cable 108 and such motion of the cable relative to the device engagement mechanism 106 may cause the device engagement mechanism 106 to close and may allow it to grasp a medical device.

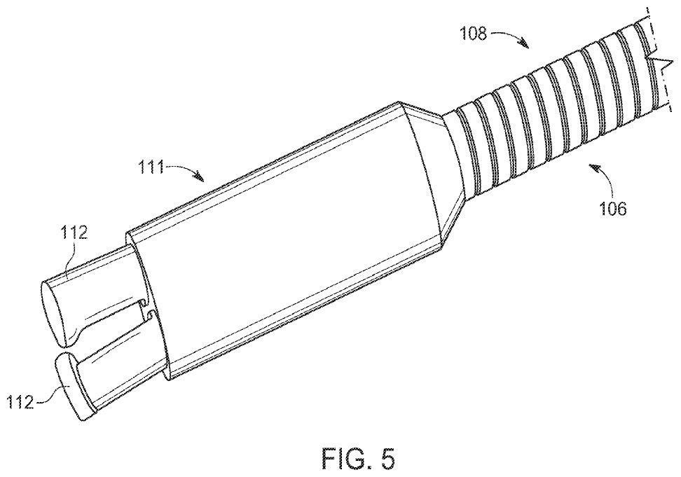

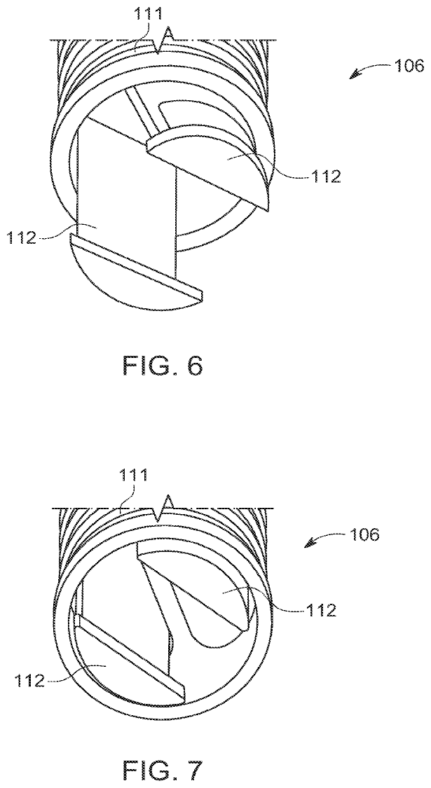

FIG. 4 is a schematic view of an example device engagement mechanism 106 arranged adjacent a medical device 15. The device engagement mechanism 106 may include prongs 112, shown in detail in FIGS. 5-7.

Referring to FIGS. 5, 6, and 7, the device engagement mechanism 106 may include a pair of prongs 112 that are connected to one another and biased in an open position. The prongs, for example, may be formed from a substantially straight bar that has been bent in half and retains a limited amount of memory such that it is biased toward an open position. The free ends of the prongs 112 may extend through and out of a collar 111 provided at a distal end of cable 108, while the end with the bend may remain within the collar or on an opposite side of the collar. Still other mechanisms may be used to allow for relative motion between the wire and the cable to actuate the engagement mechanism and grasp or release an item.

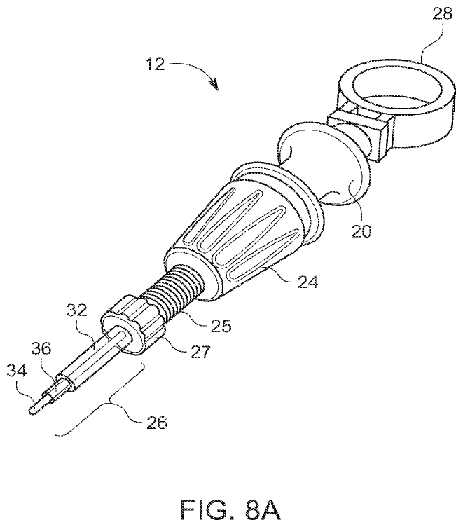

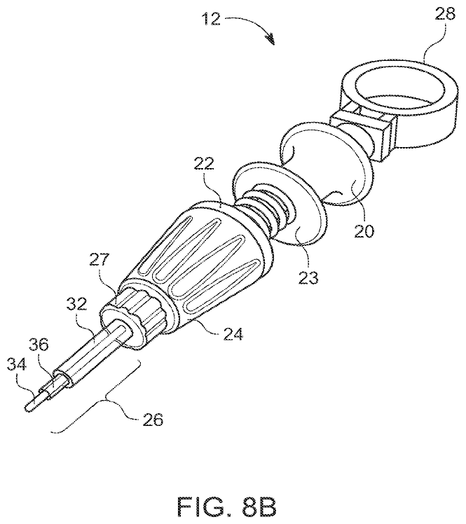

FIGS. 8a and 8b illustrate perspective views of the handle portion 12 in accordance with one embodiment. FIG. 8a illustrates the handle portion 12 in a locked position. FIG. 8b illustrates the handle portion in an unlocked position. The handle portion includes a proximal portion 20, a core 22, distal portion 24, a support member 25 having enlarged portion 27, a catheter engagement mechanism 26, and a thumb ring 28. In some embodiments, the support member 25 may be threaded. A biasing element 23, such as a spring, may be provided over the core 22 for separating the distal portion 24 from the proximal portion 20. The proximal portion 20 may also be referred to herein as an engagement control. The distal portion 24 may also be referred to herein as a locking mechanism.

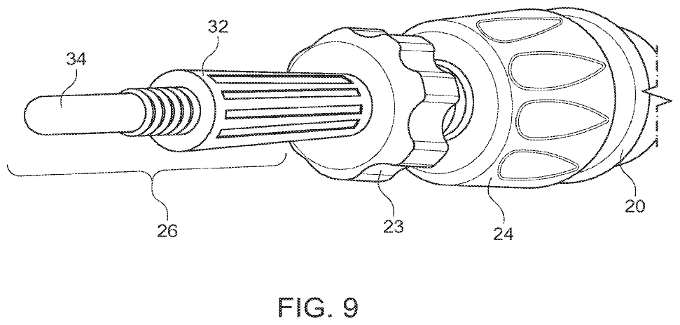

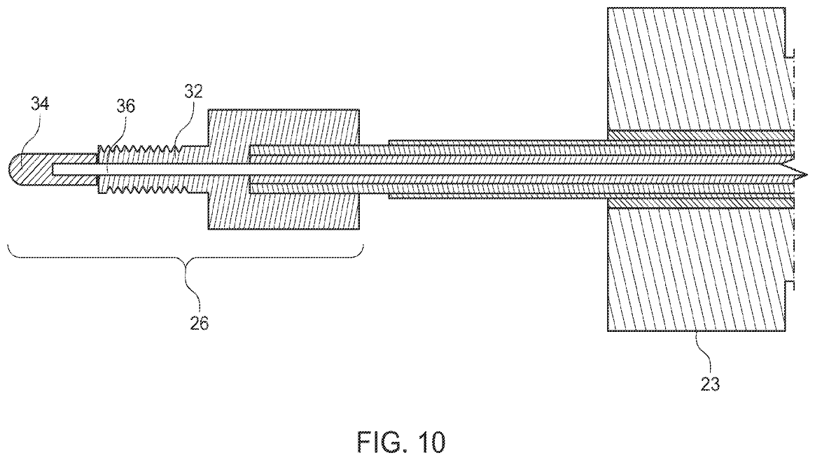

FIGS. 9 and 10 illustrate closer views of the catheter engagement mechanism 26. As shown, the catheter engagement mechanism includes a handle connector 32 and a pusher wire cap 34 at a distal end of the handle connector 32. A push wire 36 may be extended through the handle connector 32 and into the pusher wire cap 34. In some embodiments, the handle connector 32 is threaded.

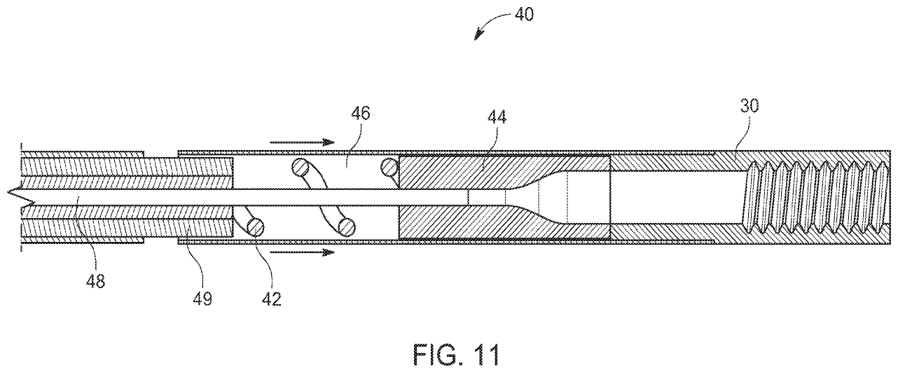

FIG. 11 is a perspective view of a proximal portion 40 of a catheter portion, in accordance with one embodiment. The proximal portion 40 of the catheter portion is configured to engage with the distal end of the handle portion. More specifically, the proximal portion 40 of the catheter portion includes handle engagement mechanism 30 for engaging the catheter engagement mechanism of the handle portion (see FIGS. 9 and 10). The handle engagement mechanism 30 and the catheter engagement mechanism may have complementary configurations for engagement. For example, the handle engagement mechanism 30 may be a female attachment mechanism and the catheter engagement mechanism may be a male attachment mechanism. Further, the handle engagement mechanism and the catheter engagement mechanism may have complementary threadings.

The proximal catheter portion 40 includes catheter shrink 49, compression spring 42, pull wire attachment 44, hypotube 46, and catheter engagement mechanism 30. A pull wire 48 may extend through the catheter shrink 49, hypotube 46, and pull wire attachment 44. The hypotube 46 extends over the pull wire attachment 44 and at least a portion of the catheter engagement mechanism 30. When the handle portion is not coupled to the catheter portion, the compression spring pushes the pull wire attachment 44, keeping the distal end of the distal end of the catheter closed. The distal end of the catheter cannot be accidentally opened, at least because the pull wire attachment 44 is full enclosed within the hypotube 46 and the handle engagement mechanism 30.

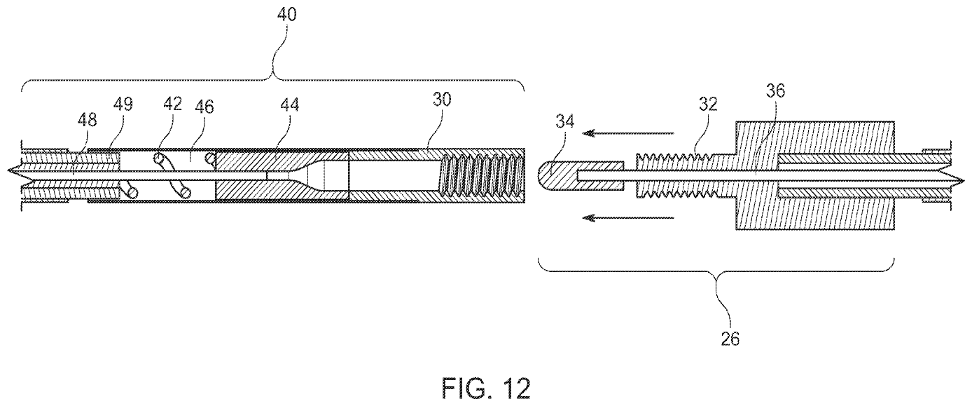

FIG. 12 illustrates the proximal catheter portion 40 of the catheter portion of the delivery device and the catheter engagement mechanism 26 of the handle portion of the delivery device pre-engagement.

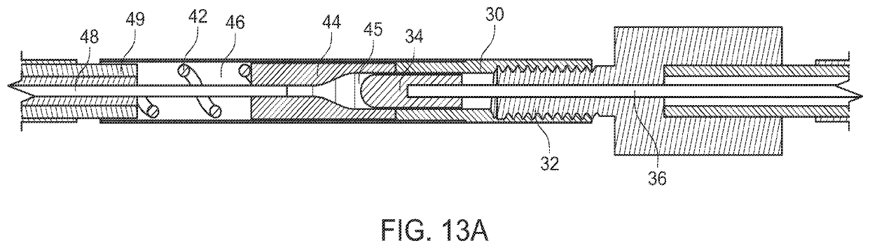

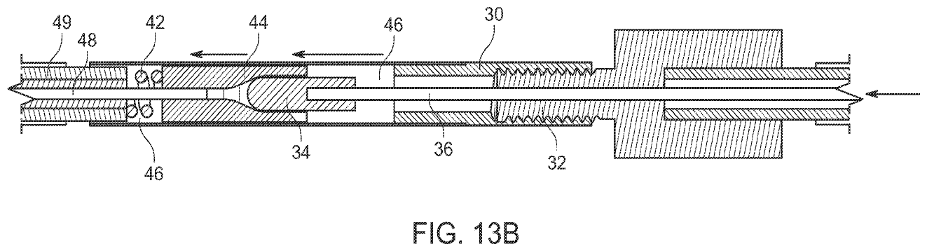

FIGS. 13a and 13b illustrate engagement of the catheter engagement mechanism of the handle portion with the handle engagement mechanism of the catheter portion, in accordance with one embodiment. FIG. 13a illustrates the compression spring 42 of the proximal portion of the catheter portion in a relaxed position, as occurs prior to deployment of the medical device. FIG. 13b illustrates the compression spring 42 of the proximal portion of the catheter portion in a compressed position, as would occur during deployment of the medical device, when the push wires 36 and pull wire 48 are forced forward. As shown, when the proximal catheter portion 40 engages the catheter engagement mechanism 26, the pusher wire cap 34 of the catheter engagement mechanism 26 is received by a complementary receptacle 45 in the pull wire attachment 44 of the proximal portion 40 of the catheter portion. Similarly, the handle connector 32 of the catheter engagement mechanism 26 is received by the handle engagement mechanism 30 of the proximal portion 40 of the catheter portion. The handle connector 32 may be releasably fixed in engagement with the handle engagement mechanism 30, for example via threading.

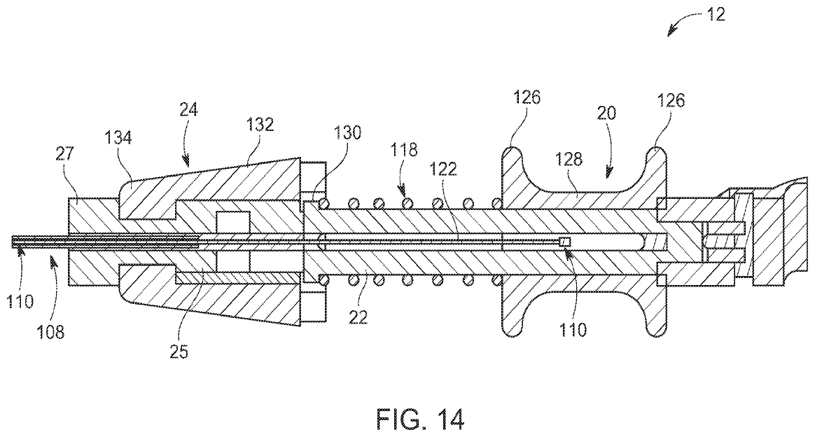

An understanding of the operation of the handle portion relative to the device engagement mechanism may be helpful for a better understanding of the splice mechanism discussed below. FIG. 14 illustrates a handle portion 12, in accordance with one embodiment. As shown, the handle portion 12 may include a several operable elements for manipulating the device engagement mechanism at the distal end of the delivery system. For example, the handle portion 12 may include a proximal portion 20, a core 22, and distal portion 24. A biasing element 118 may be provided over the core 22 for separating the distal portion 24 from the proximal portion 20. The proximal portion 20 may be referred to herein as an engagement control. The distal portion 24 may be referred to herein as a locking mechanism.

The proximal portion 20 includes flanges 126 and a crossing member 128. The core 22 includes a slot 122 and a biasing element 118. The distal portion 24 includes a shoulder 130, a body 132, and a nut portion 134. A cable 108 with a wire 110 may extend through the handle portion to an embedded position, described more fully below.

The core 22 of the handle portion 12 may be configured for receiving the cable/wire at a distal end thereof and for allowing relative motion between wire portion of the cable/wire and the core. The core 22 may be secured to the cable portion of the cable/wire and may generally resist relative motion between the core and cable. The core may include a generally tubular structure having an inner lumen for receiving the wire 110 and may include an outer peripheral surface for mounting or arrangement of the proximal portion 20, the biasing element 118, and the distal portion 24. The core may include a slot or window 122 extending along the length of the core 22 to provide access to the wire within the core. The core may have a thumb ring 124 or other element at a proximal end for engagement with the thumb of a user, for example.

The proximal portion 20 may be mounted on the core and may be articulable relative to the core to cause and/or control movement of the wire 110 relative to the core/cable and, thus, control the engagement mechanism 106. In one or more embodiments, the engagement control may include a collar arranged on the core that is configured for sliding back and forth along the core. As shown, the collar may have a spool shape with an inner bore for receiving the core and an outer surface with a flange 126 at each end. The spool may be sized and configured for positioning between two figures of a user allowing the collar to be conveniently operable along the core. The collar may include a crossing member 128 extending across the bore for passing through the window of the core for engaging the wire, such that longitudinal motion of the engagement control causes longitudinal movement of the wire relative to the core and the cable. As shown in FIG. 14, the wire may be embedded in the crossing member, for example.

The biasing element 118 may be arranged on the core and may be configured to bias the engagement control in a particular direction. For example, the core may include a circumferential rib or shoulder 130 at or near a proximal end of the window 122. The biasing element 118 may include a spring, for example, that is seated on the shoulder 130 and that biases the engagement control in a proximal direction thereby maintaining tension on the wire and causing the engagement mechanism at the distal end to be biased in a closed or clamping position. As such, to release a medical device from the engagement mechanism, the engagement control may be pressed forward or distally against the biasing force of the biasing element and when the biasing force is overcome, the engagement control may move forward or distally along the core, thereby advancing the wire relative to the cable and releasing the clamping force of the engagement mechanism.

The distal portion 24 may be arranged on the core and may be configured to prevent inadvertent disengagement of the engagement mechanism due to inadvertent advancement of the engagement control. While a distal portion is specifically shown and described, it is to be appreciated that any suitable andi-release mechanism may be used to prevent inadvertent release of the medical device. For example, when the medical device is engaged by the delivery system prior to placement in the patient, the locking mechanism may be used to secure the position of the engagement control device to prevent or reduce the likelihood of inadvertent release of the medical device. As shown in FIG. 14, the distal portion 24 may include a threaded nut 134 or multiple threaded nuts that may be backed against the engagement control to prevent advancement thereof. The outer surface of the core may include a threaded surface arranged distally to the biasing element shoulder. The threaded surface may allow the locking mechanism to be rotated causing the locking mechanism to move proximally relative to the core to a position abutting the distal end of the engagement control. The locking mechanism may include a body 132 with a bore sized to sleeve over the biasing element and a threaded nut portion 134 at a distal end for engaging the threads. The locking mechanism may be rotated causing the threaded nut portion to walk proximally along the core toward the biasing element shoulder. The body/bore portion of the locking mechanism may encompass the shoulder and the biasing element as the locking mechanism is moved in the proximal direction to engage the engagement control. A locking nut may be provided distal to the body to provide a measure of protection against inadvertent loosening of the body, which could lead to inadvertent advancement of the engagement control. In other embodiments, an extension screw may be provided on the proximal end of the body that may threadingly extend from the proximal end of the body thereby providing engagement with the engagement control and a resistance to inadvertent loosening of the locking mechanism.

A support member 25 with an enlarged portion 23 may be provided for receiving the distal portion 24. The distal portion 24 may be threaded distally along the support member until it abuts the enlarged portion 27. In this position, the handle portion 12 may be unlocked.

Returning now to FIG. 1b, it is to be appreciated that the delivery system 10 permits the handle portion 12 to be decoupled from the catheter portion 14. As shown, the system may include an operable handle 12 and a cable/wire extending away from the handle portion 12 to a device engagement mechanism 18 at a distal end. The catheter portion 14, and more specifically, the cable/wire, may be decoupleable from the handle portion 12, allowing the handle portion 12 to be removed. This decouplability may be provided by a series of elements within or around the handle that allow the cable/wire to be disconnected from the handle while the relative position of the cable and wire are maintained. That is, tension on the wire may be maintained throughout the process of decoupling so that constant and secure control over the medical device at the distal end is maintained.

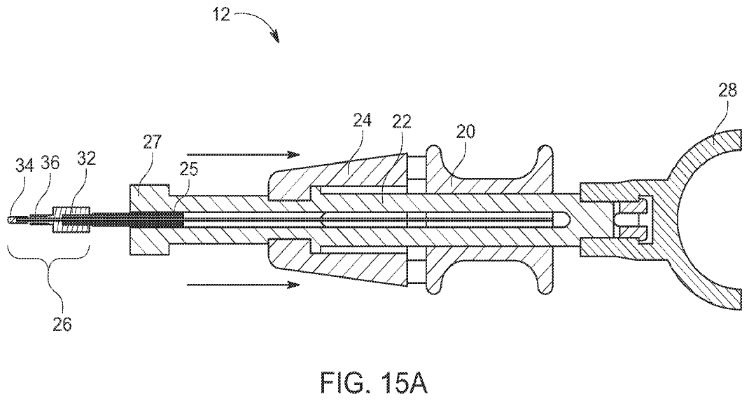

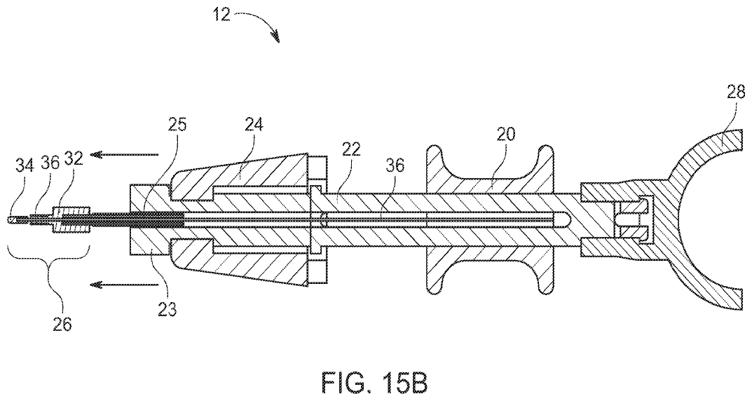

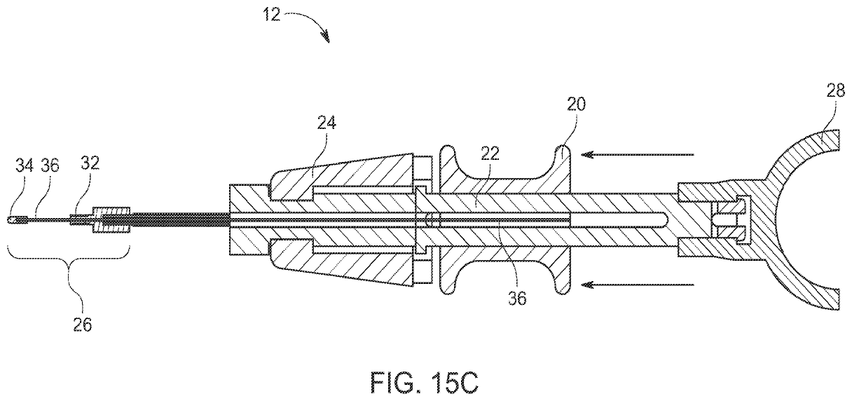

FIGS. 15a-15c illustrate the handle portion 12 and catheter engagement mechanism 26, in accordance with one embodiment. As shown, the catheter engagement mechanism 26 includes a handle connector 32 and a pusher wire cap 34 at a distal end of the handle connector 32. The handle portion 12 includes a proximal portion 20, a core 22, a distal portion 24, and a thumb ring 28. A push wire 36 extends from the proximal portion 20 through the core 22, the distal portion 24, and the handle connector 32, and into the pusher wire cap 34. A support member 25 supports the distal portion 24. An enlarged portion 27 limits distal movement of the distal portion 24 along support member 25.

FIG. 15a illustrates the handle portion 12 in a locked position with the distal portion 24 of the handle portion 24 fully backed up to the proximal portion 20. Movement of the distal portion 24 to the position may be done by threading of the distal portion along support member 25.

FIG. 15b illustrates the handle portion 12 in an unlocked position. In this position, the distal portion 24 is forward of the proximal portion 20 with a gap created along the core 22. The distal portion 24 is at its forwardmost position along support member 25 abutting enlarged portion 27 of support member 25. Movement of the distal portion 24 to this position may be done by unthreading the distal portion along support member 25.

FIG. 15c illustrates the handle portion 12 in an unlocked position with the catheter distal end opened. In this position, the distal portion is at its forwardmost position along support member 25. The proximal portion 20 is moved forward towards the distal portion 24. Movement of the proximal portion 20 to this position is achieved by pushing the proximal portion 20 forward. This may be done against the force of a biasing member such as shown in FIG. 8b.

Returning to FIGS. 1b and 13a, the delivery device 10 is assembled by threading handle connector 32 of catheter engagement mechanism 26 of handle portion 12 is threaded into handle engagement mechanism 30. The pusher wire cap 34 is then inserted through handle engagement mechanism 30 and into receptacle 45 of pull wire attachment 44. Assembly will typically be done with the handle portion 12 in the locked position shown in FIG. 8a. In this position, the handle portion 12 is fixed to the catheter portion 14 and the delivery device 10 is assembled.

Upon assembly of the device 10, the handle portion 12 may be moved into the unlocked positon of FIG. 8b by threading distal portion 24 along the support member until it abuts the enlarged portion 27. The proximal portion 20 of the handle portion 12 may then be pushed forward along the core 20 to pusher wire cap 34 forward, compressing spring 42 in hypotube 46. In this position, the device engagement mechanism 18 can be actuated to close prongs 116 to grasp medical device 15. Upon engagement of the medical device 15, the handle portion may be moved back into the locked position.

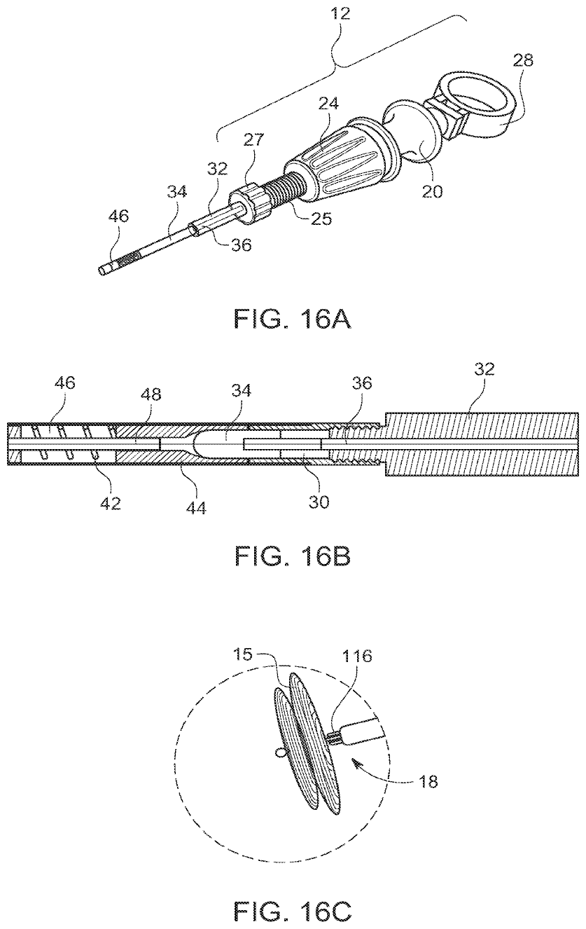

FIGS. 16a-16c illustrate the delivery device 10 with the handle portion 12 in a locked position, such as shown in FIG. 15a, the handle portion coupled with the catheter portion such as shown in FIG. 13a, and the medical device 15 grasped by the device engagement mechanism 18. In this position, the distal portion 24 of the handle portion 12 is full retracted against the proximal portion 20 of the handle portion 12, such as by threading the distal portion 24 along the support member 25.

FIG. 16a illustrates the handle portion 12 and the proximal end of the catheter portion. FIG. 16b illustrates a cross sectional view of the engagement of the cather connection mechanism with the handle connection mechanism. Prongs 116 are manipulated to close on an attachment mechanism of the medical device 15. FIG. 16c illustrates the distal end of the catheter portion with the medical device 15 grasped by the delivery device.

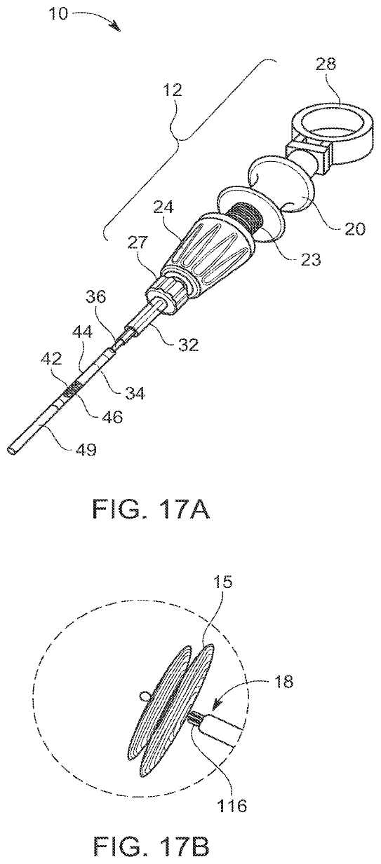

FIGS. 17a and 17b illustrate the delivery device 10 with the handle portion 12 in an unlocked position, such as shown in FIG. 15b, and the device engagement mechanism 18 engaging medical device 15. FIG. 17a illustrates the handle portion 12 and a proximal portion of the catheter portion. FIG. 17b illustrates the distal end of the catheter portion and medical device 15. In the position of FIG. 17a, the catheter engagement mechanism 26 of the handle portion 12 is engaged with the handle engagement mechanism of the catheter portion. The pusher wire cap 34 of the catheter engagement mechanism 26 is inserted in the pull wire attachment 44. The handle connector 32 is threaded into the catheter threaded insert 30.

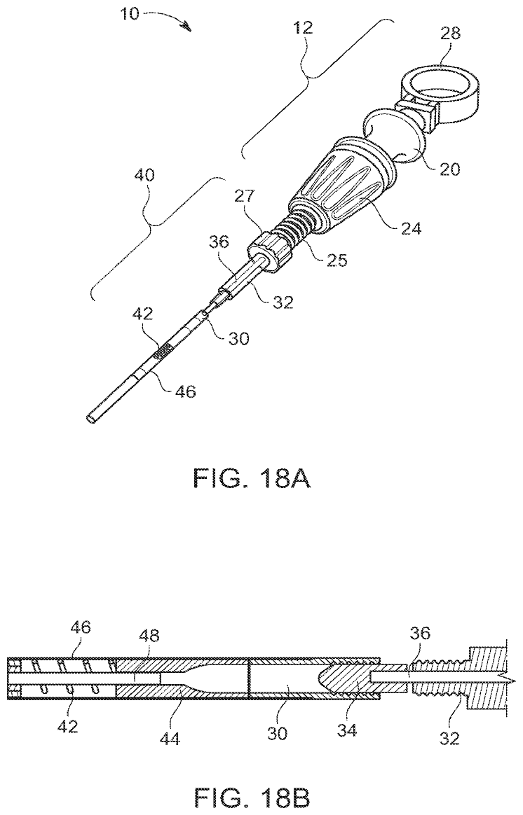

FIGS. 18a and 18b illustrate the delivery device 10 with the handle portion 12 in a locked position, such as shown in FIG. 15a, and the proximal catheter portion 40 of a catheter portion and a catheter engagement mechanism of a handle portion being decoupled. Decoupling may be done to facilitate insertion of a lengthening component such as a wire extension.

FIG. 18a is a perspective view of a handle portion and a proximal portion of a delivery device with the handle portion in a locked position. FIG. 18b is a cross-sectional view of a proximal catheter portion 40 of a catheter portion and a catheter engagement mechanism of a handle portion, with the catheter engagement portion of the handle portion being withdrawn from the catheter portion. More specifically, in the position of FIGS. 18a and 18b, the handle connector 32 has been unthreaded from the catheter threaded insert 30 and the pusher wire cap 34 is being pulled from the pull wire attachment 44 through the threaded insert. Clear separation exists between push wire 36 and pull wire 48. It is to be appreciated that the positioning of the device portions in FIGS. 18a and 18b is the same pre-engagement and post-engagement and decoupling.

In this position, the compression spring 42 is pushing pull wire attachment 44, keeping the distal end of the catheter closed. The distal end cannot be accidentally opened because the pull wire attachment 44 is fully encased in the hypotube 46 and the threaded insert 30. Thus, even with the handle portion decoupled from the catheter, the medical device 15 is maintained in position relative the catheter portion 14.

Once the handle portion 12 has been removed, the sheath of the catheter portion may be exchanged, the wire within the catheter portion may be lengthened, or the catheter portion may be otherwise manipulated.

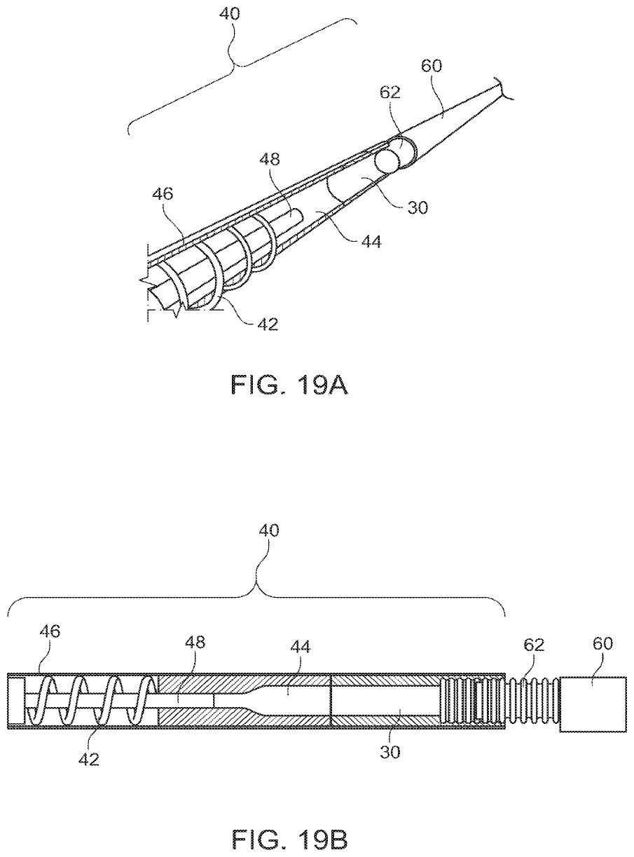

FIGS. 19a and 19b illustrate cross sectional views of cable to cable operation. A cable 60 having a threaded end 62 is inserted and threaded into the handle engagement mechanism 30 of the proximal catheter portion 40. When the cable 60 is engaged, the catheter may be exchanged and/or a guidewire extension may be added.

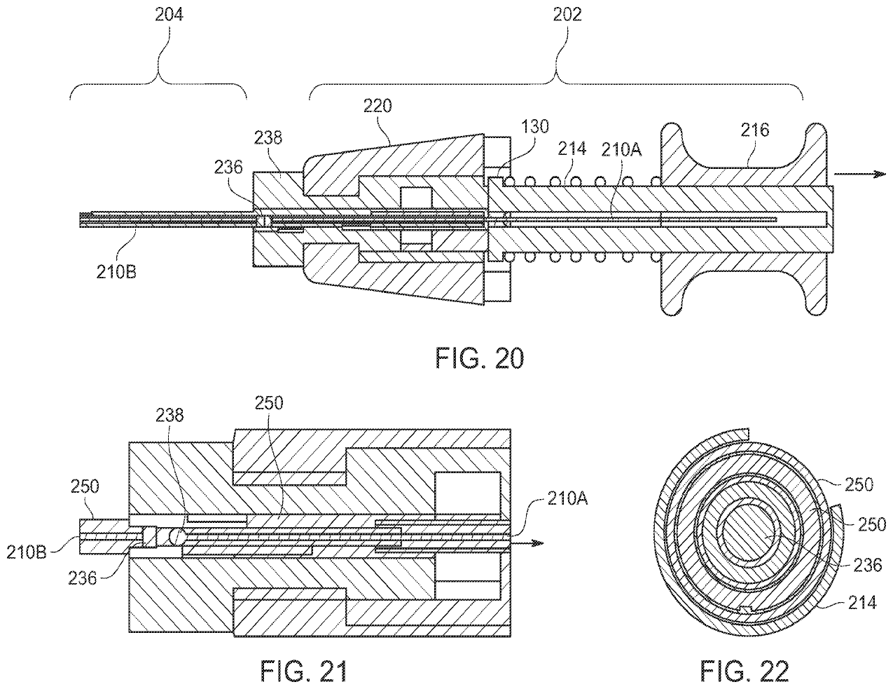

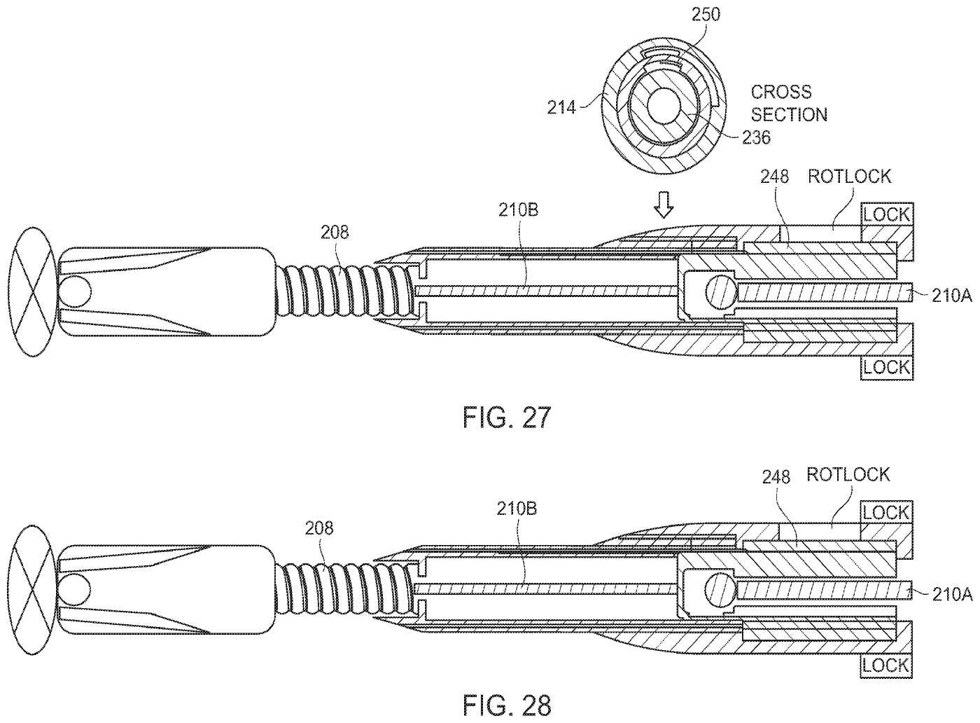

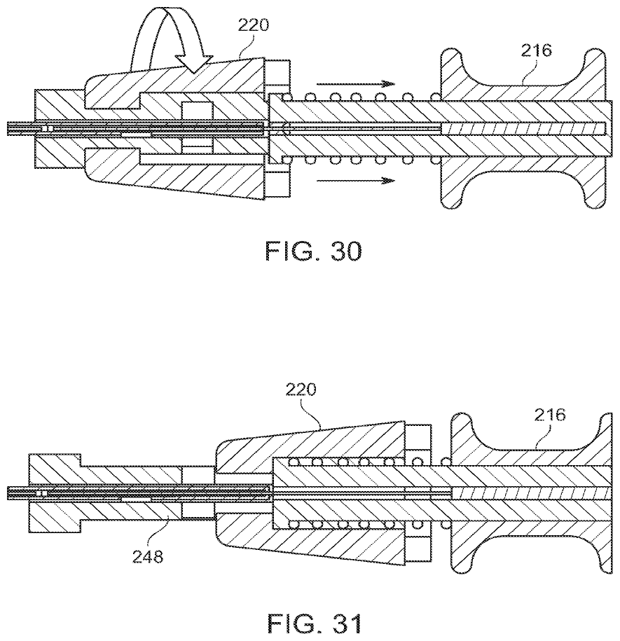

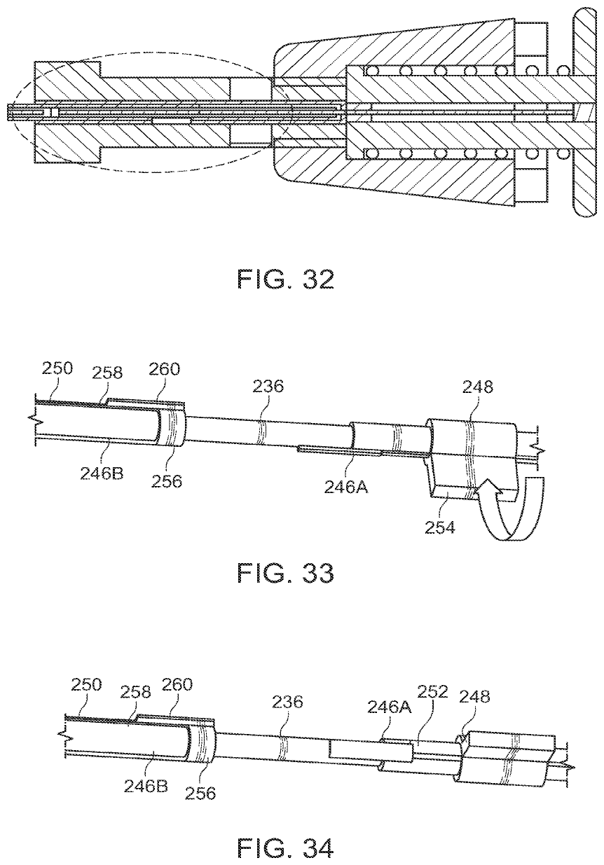

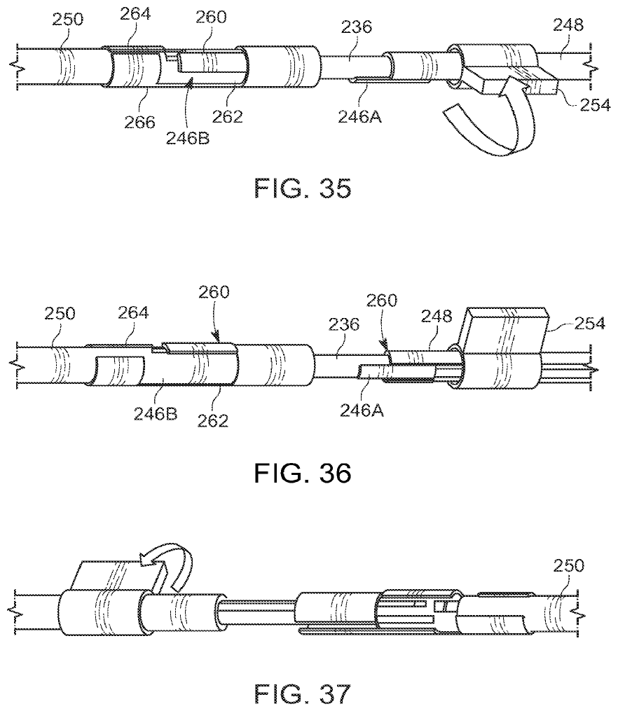

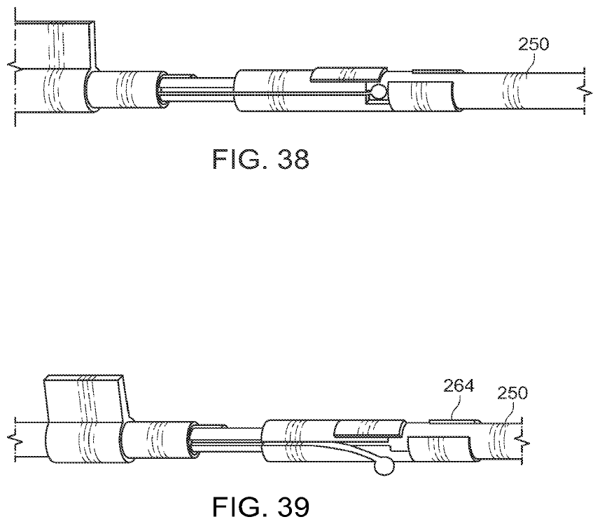

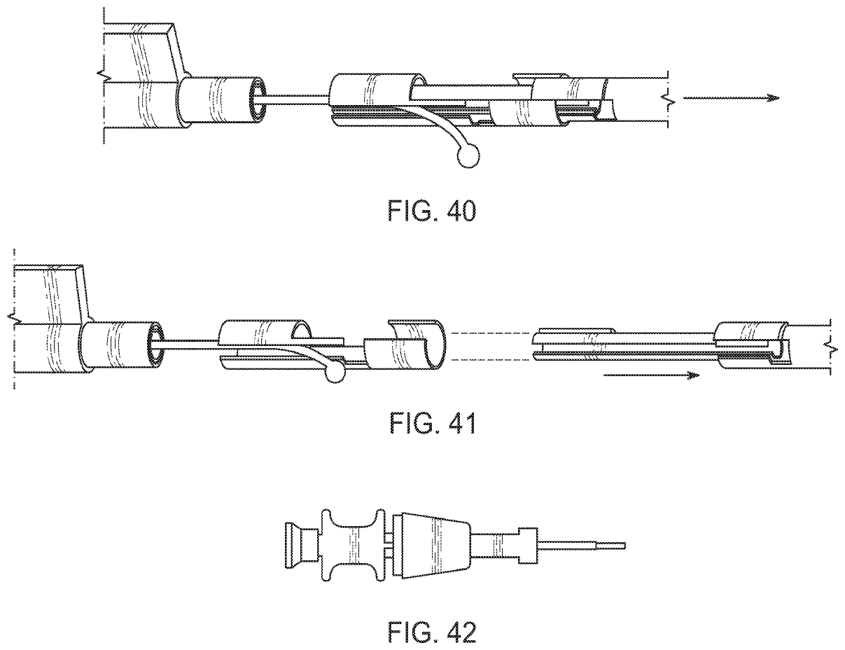

FIGS. 20-44 illustrate an alternative embodiment of a decouplable delivery system. It is to be appreciated that like structures of embodiments described above and embodiments described below may operate in the same manner and the description provided applies to each. As shown in FIG. 20, and in close up in FIG. 21, the wire 210 may include a proximal portion 210A that is secured to the engagement control in a manner similar to that describe above. However, the wire 210 may include a distal portion 210B as well and a coupling element 236 may be provided for securing the distal and proximal portions of the wire. As shown, the proximal portion 210A of the wire 210 may be secured to the engagement control 216 at one end and may include a bulb, ball, Tee, or other enlarged feature 238 for secured engagement with the coupling element 236 at the other end. The proximal portion may also have a naturally curved shape. That is, when the proximal portion of the wire is held within the system, the proximal portion may be held in a straight arrangement, but may otherwise have a memory causing it to be naturally curved and thus having a tendency to press the enlarged end 238 laterally against the inner surface of coupling element 236. The distal portion 210B of the wire 210 may be secured to the engagement mechanism 206 at the distal end of the system and may be fused, welded, pinned, or otherwise substantially permanently secured to the coupling element 236.

One embodiment of a coupling element 236 is shown in FIG. 24A. (Note: its orientation is 180 degrees as compared to the orientation in FIGS. 10-13.) As shown, the coupling element 236 may include substantially tubular structure configured for arrangement within a lumen of the core 214. The coupling element 236 may include a longitudinally extending slot 240 on a side thereof. The slot 240 may extend from a proximal end of the coupling element and along a large majority of the coupling element to a release window 242. The coupling element 236 may include a release window configured for allowing the enlarged end 238 of the proximal portion of wire to release out the side of the coupling element when the window is oriented such that the window is open or uncovered. As shown in FIG. 39, when such an orientation is provided, the enlarged end of the proximal portion of the wire and the portion of the wire extending through the coupling element may be releasable out the side of the coupling element through the slot and the window. The coupling element may also include an insert 244 for arrangement within the tubular structure causing a restriction in the internal diameter of the coupling element at a position proximal to the window. The restriction in the internal diameter of the coupling element may provide a stop against which the enlarged portion of the wire may seat when the wire is under tension.

As shown in FIGS. 33-41, the coupling element 236 may also include one or more external position alignment guides 246A/B. The guides 246A/B may protrude from an outside surface of the coupling element 236 on a side of the coupling element generally opposite the slot/window. The guides may extend longitudinally along the outside surface of the coupling element and may have parallel extending edges for aligning with and engaging a slot in a concentrically arranged element. One of the guides may be a proximal guide 246A and it may be configured for engaging a release element 248. The other guide may be a distal guide 246B and it may be configure for engagement with a cable tying element 250 to resist and/or prevent relative motion between the wire and the cable in particular circumstances.

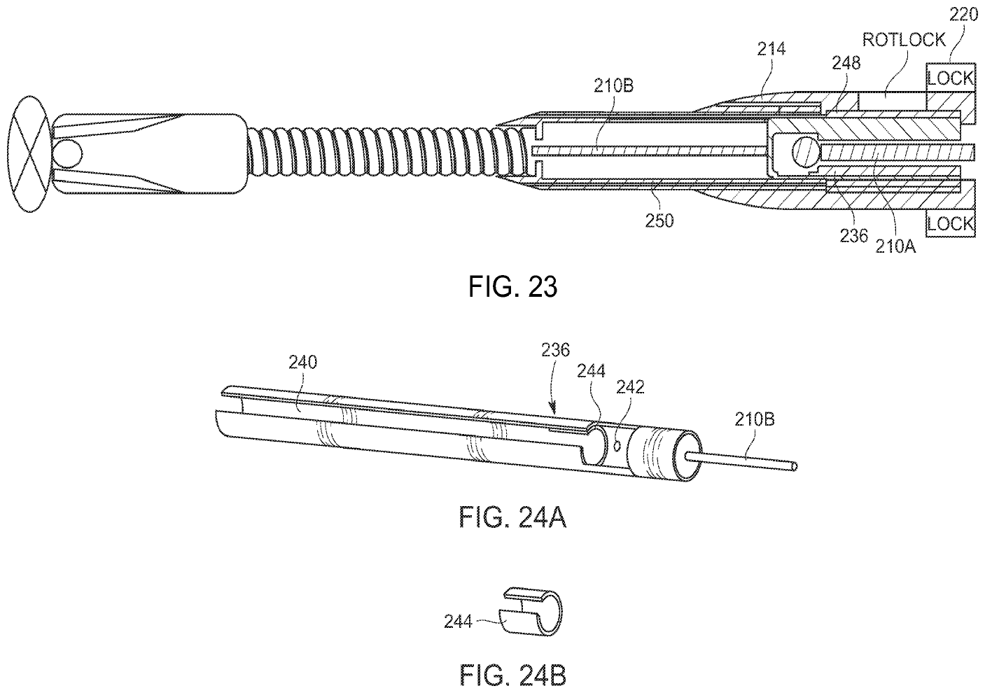

As described in more detail below, a series of other concentrically arranged elements may be provided for controlling the relative motion of the coupling element 236 and the cable when the handle is removed. As shown in FIGS. 23-31, for example, and in addition to the handle core 214 and the coupling element 236, a release element 248 and a cable tying element 250 may be provided. The core element 214 may be the outer most concentric tube. The release element 248 and the cable tying element 250 may have the same or similar diameters and may be arranged within the core element 214. As also shown, the coupling element 236 described above, may have a diameter allowing for arrangement within each of the release element 248 and the cable tying element 250.

The release element 248 may be configured for controlling the engagement of the several concentric elements and allowing for decoupling of the handle. As shown in FIG. 31, when the locking element 220 of the handle is positioned in a locking position (i.e., proximally located against the engagement and control element 216), the release element 248 may be exposed distal to the locking element 220. FIGS. 23-31 omit the handle elements other than the core element for clarity. A shown, the release element 248 may be a tubular structure arranged generally over the proximal portion of the coupling element 236 and having a slot 252 for receiving the proximal guide element 246A on the outer surface of the coupling element 246. In particular, during operation of the handle, the proximal guide element 246A of the coupling element 236 may be relatively free to articulate within the slot 252 of the release element 248, while rotational motion between the coupling element and the release element may be prevented. The release element may also include a lever 254 extending laterally from the tubular body and through a portion of the core to expose the lever to the user and allow manipulation thereof.

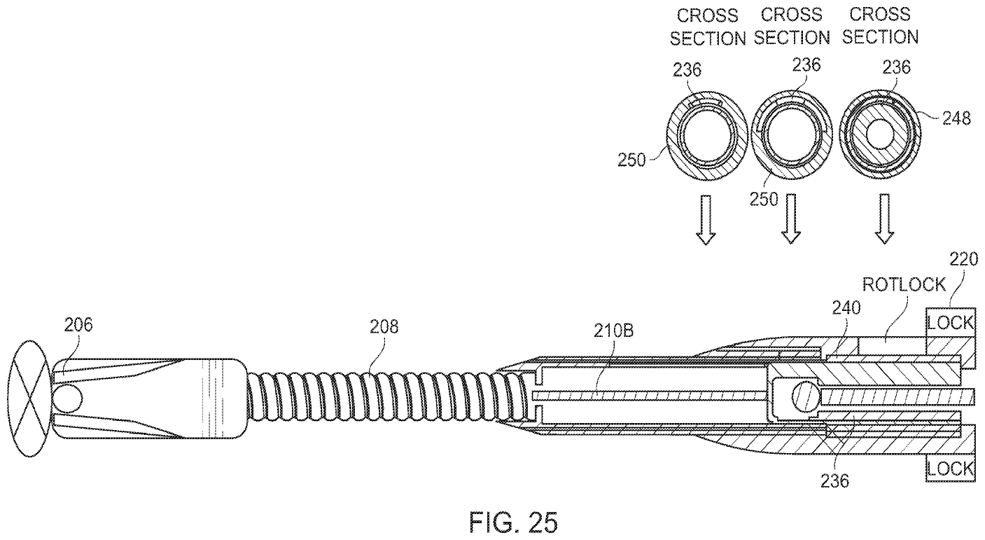

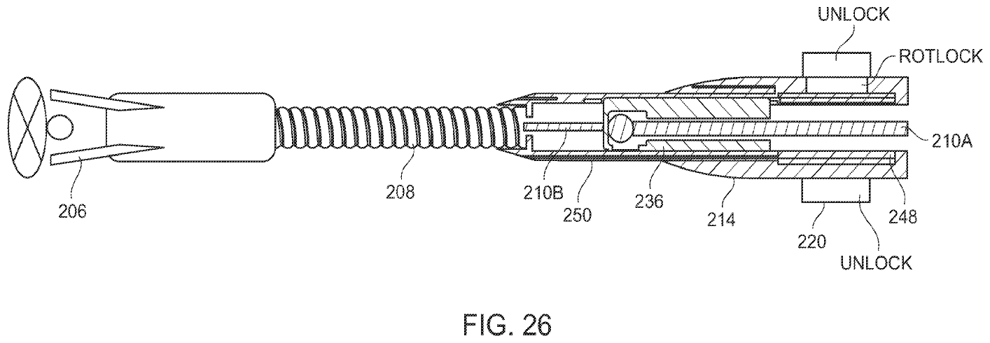

The cable tying element 250 may be configured for allowing free longitudinal articulation of the coupling element 236 unless the handle is being removed at which time, the cable tying element is configured to engage the coupling element 236 and prevent relative longitudinal motion of the cable and the wire. The cable tying element 250 may be concentrically arranged within the core element 214 and concentrically around the distal portion of the coupling element 236. The distal end of the cable tying element may be fixed to the proximal end of the cable 208. The cable tying element 250 may also include a slot 256 for articulably receiving the distal guide 246B of the coupling element 236 and allowing the coupling element 236 to articulate along the slot 256. The cable tying element 250 may also include an adjacent slot or keyway 258 for receiving the distal guide 246B of the coupling element 236 when the coupling element is rotated relative to the cable tying element 250. That is, as shown in FIGS. 23 and 24, when the lever 254 of the release element 248 is rotated, the coupling element 236 may rotate causing the distal guide 246B to engage the keyway 258 of the cable tying element 250. Engagement of the distal guide 246B with the keyway 258 of the cable tying element 250 may prevent relative longitudinal motion between the cable tying element and the coupling element and, thus, the cable and the wire. As such, this engagement may allow for maintaining constant secure control over the engagement mechanism. Like the coupling element, the cable tying element may include an external control and alignment guide 260 at a proximal end thereof. The proximal alignment guide 260 on the cable tying element 250 may be configured for engagement with the core element 214 to maintain engagement of the cable 208 and the handle 202 unless and until it is to be released. As shown in FIGS. 25 and 26, the proximal guide 260 may engage a keyway 262 on the core element 214 which may prevent relative longitudinal motion between the cable tying element 250 and the core element 214. However, upon rotation of the cable tying element 250 within the core element 214, the proximal guide 260 of the cable tying element may align with a release slot of the core element 214 allowing the cable tying element 250 to be released from the core element 214 and the handle 202 while still secured to the coupling element 236.

It is to be appreciated that while a separate core element is shown in FIGS. 25-30, the core element 214 previously described may include grooves and slots on an inner surface thereof providing for the engagement with the cable tying element 250 in the manner shown and described. As shown, the core element 214 may include a longitudinal slot 264 extending out a distal end thereof for releasing the cable tying element 250 when the proximal guide on the cable tying element is aligned with the slot. The core element 214 may also include a keyway 266 alongside the slot 264 for engagement of the proximal guide 260 on the cable tying element 250 and preventing relative longitudinal motion of the cable tying element.

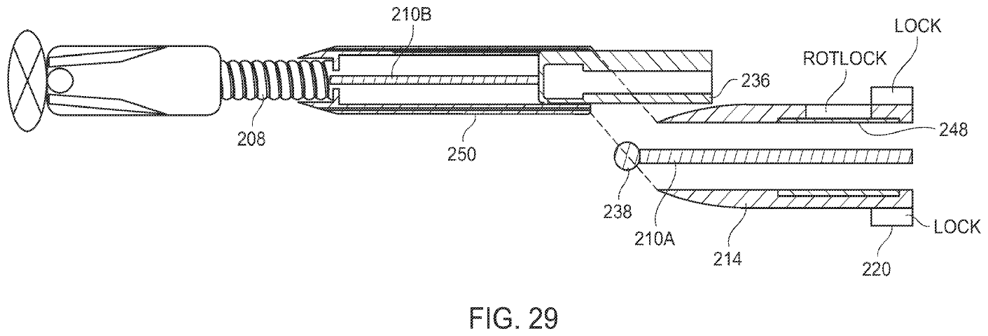

As shown in FIGS. 28 and 29, the cable tying element 250 and the core element 214 may include a slot and window similar to that of the coupling element 236. The slot and window in these elements may be arranged such that when the coupling element 236 is rotated to engage the cable tying element 250 and the cable tying element 250 is rotated to a release position (i.e., the proximal guide 260 of the cable tying element 250 is aligned with the slot in the core element), each of the respective slots and window all align allowing the proximal portion 210A of the wire and its enlarged portion 238 to spring free from the several elements. That is, the memory of the proximal portion 210A of the wire being a curved shape may cause the wire to spring free from the elements when the slot and windows of the several elements are aligned. In the core element, the window may be aligned with a recess sufficient to receive the enlarged portion of the wire thereby allowing the enlarged portion to fully exit the coupling element thereby releasing the coupling element and allowing the slot of the coupling element to slide along the exiting wire without resistance by the enlarged portion. In one or more embodiments, as shown in FIG. 42, the recess may extend through the sidewall of the core element 214 providing visual confirmation of the wire being released and also allowing for access to the wire for reattachment to the coupling element at a later time.

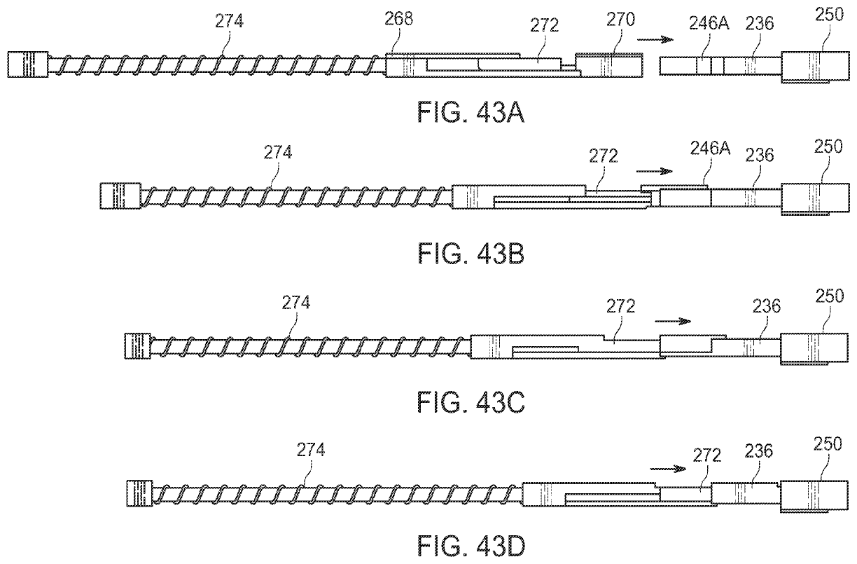

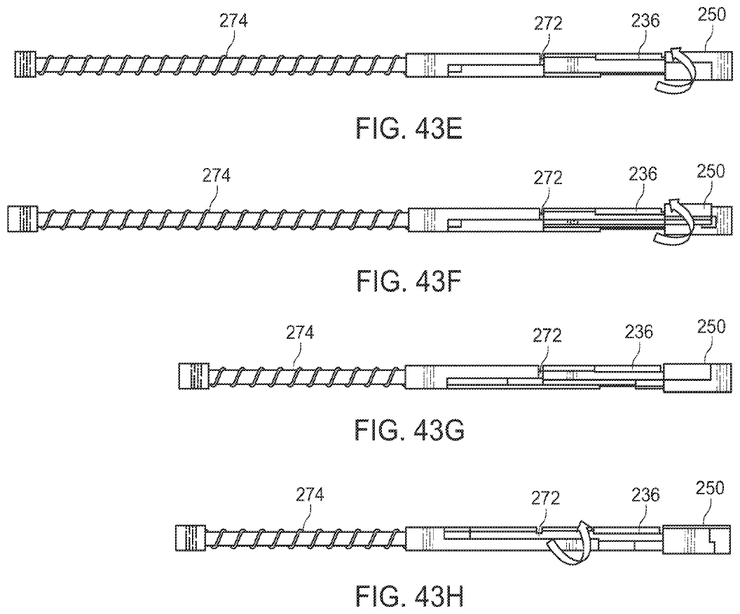

Turning now to FIGS. 43A-43H, an extension element 268 is shown. The extension element 268 may be configured for attachment to the coupling element 236 to extend the length of the system outside the patient when the handle has been removed. The extension 268 may allow the sheath to be removed without letting go or otherwise relinquishing control of the delivery system in the patient. The extension element 236 may include a distal end configured for sleeving over and engaging the coupling element 236. As such, the extension element 268 may include a distal end having a tubular structure for receiving the coupling element and may include a slot 270 for receiving the proximal guide 246A of the coupling element 236. The extension element may also include a keyway 272 such that once the extension is sleeved over the proximal end of the coupling element, the extension may be rotated causing the proximal guide 246A on the coupling element to engage the keyway 272 and prevent relative longitudinal motion of the extension element and the coupling element. In addition, the coupling element may include a biasing element 274 such as a spring to cause longitudinal compression and/or tension between the extension element 268 and the coupling element 236 thereby resisting rotation by way of friction and avoiding inadvertent disconnection. The extension element may be substantially long including lengths substantially equal to the sheath length or slightly longer allowing the sheath to be removed from the patient fully without reaching the proximal end of the extension element and, thus, allowing full removal of the sheath and access to the coupling element before the release of the extension element is needed to allow the sheath to be removed from the extension element.

In operation, the present delivery system may be used to deliver a medical device to a deployment location within a patient. The system may be advantageous when a sheath becomes damaged or when, after steps have been taken to deliver a medical device, the user wishes to retrieve it, but is not able to pull the device into the sheath because the sheath is too small (i.e., when the medical device has been expanded after exiting the sheath, but will not collapse back into its delivery condition). In either of the above situations, the user may wish to remove the sheath without removing the delivery system. The present delivery system may be advantageous because it may allow for decoupling a proximal handle from the system, while maintaining control over the system and, in particular, the medical device secured to the distal end of the system.

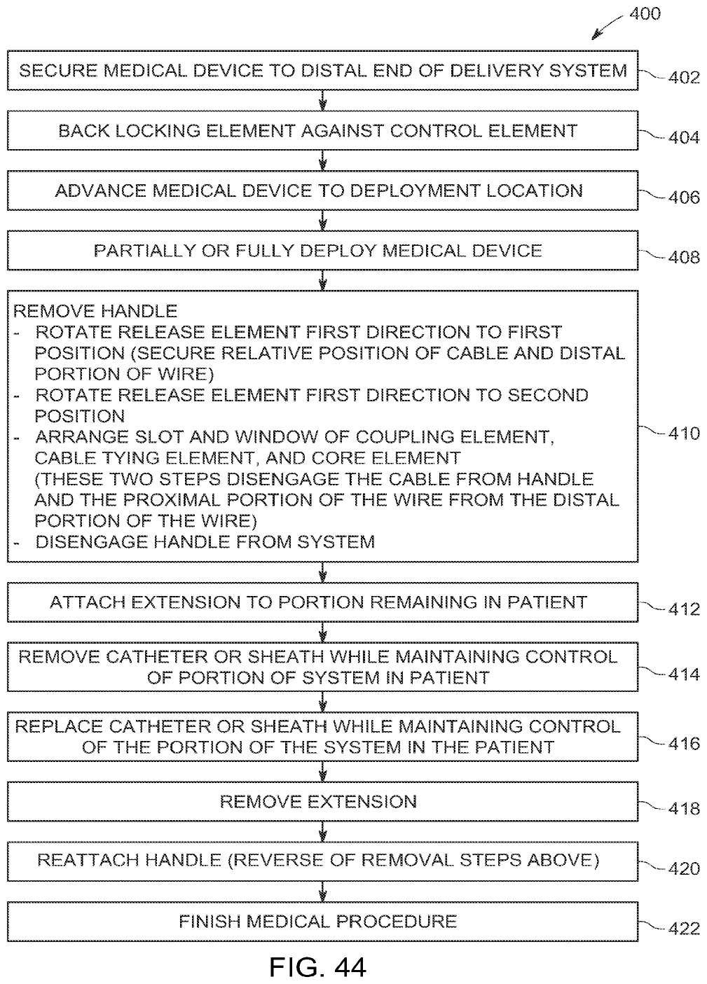

As shown in FIG. 44, in one or more embodiments, the system may be used in a method 400 to deploy a medical device such as a device for treating or closing an atrial septal defect. The delivery system may be connected to the medical device (402) by grasping a bulb, pin, or other graspable element with the engagement mechanism on the distal end of the delivery system. The handle on the system may be used to grasp the medical device by advancing the engagement or control element against the force of the biasing element to cause the engagement mechanism to open. The graspable element on the medical device may be placed between prongs of the engagement mechanism and the engagement or control element may be released allowing the biasing element to retract the control element and the wire relative to the cable thereby closing the prongs on the device. To reduce the risk of inadvertent release of the medical device, the locking element on the handle may be backed over the biasing element against the control element to resist and/or prevent advancement of the control element along the handle (404).

With the medical device attached, the delivery system may be used to advance the medical device through a sheath to a deployment location such as that shown in FIG. la, for example (406). Upon reaching the deployment location, the system may be used to advance the medical device out of the end of the end of the sheath to deploy the medical device (408). The distal end of the sheath may be strategically placed such that deployment of a portion of the device occurs in the left atrium and then the sheath may be backed off causing the remaining portion of the device to be deployed in the right atrium. This may allow the device to suitably sandwich the atrial septum while occluding a hole in the atrial septum. In some cases, however, the placement of the device may not be ideal, it may be incorrect, or it may otherwise be unsuitable as delivered. Due to the self-expanding nature of the medical device, it may not readily be pulled back into the sheath for removal. In these circumstances, it may be desirable to remove the sheath and replace it with a larger one more capable of receiving the expanded medical device. In still other cases, the sheath may be damaged and it may be desirable to remove the damaged sheath.

In these and other circumstances, the handle of the present system may be removed to allow the sheath to be removed (410). With the locking element backed against the control element, the lever of the release element may be exposed on a surface of the handle as shown in FIG. 31. A user may rotate the release element to begin the process of decoupling the handle by rotating the release element in a first rotational direction to a first rotational position. Rotating the release element may cause rotation of the coupling element and, in turn, causing the coupling element to engage the cable tying element. Having engaged the cable tying element, the coupling element may no longer be free to move longitudinally relative to the cable tying element and, as such, actuation of the engagement mechanism at the distal end of the system may be resisted or prevented. With the coupling element engaged with the cable tying element, the release element may be rotated further in the same direction and to a second position causing the cable tying element to disengaged from the core element. Moreover, this further rotation may bring the slot and window structures of the coupling element, the cable tying element, and the core element into alignment. The proximal portion of the wire may spring free from the coupling element. That is, the memory of the proximal portion of the wire may cause the enlarged end of the proximal wire to move laterally through the window of the coupling element, the cable tying element, and the core element. Once the enlarged end has moved laterally through the window of the coupling element and the cable tying element, the system may be decoupled such that the medical device, the engagement mechanism, the cable, the distal portion of the wire, the cable tying element, and the coupling element may be separated from the handle and the core and release elements thereof, thereby removing the handle from the portion of the system remaining in the patient.

As may be appreciated, having removed the handle, the proximal portion of the system remaining in the patient may extend out of the patient a relatively short distance. As such, were a user to attempt to remove the sheath at this point, the user would have to release any grasp or control over the coupling element and the cable tying element to allow the sheath to slide over it. In an effort to maintain continuous control over the system, an extension may be secured to the portion of the system remaining in the patient (412). An extension system may, thus, be secured to the coupling element by advancing a distal end of the extension over the coupling element and twisting the two elements relative to one another to engage them. A biasing element may be depressed to cause the guide on the coupling element and the keyway on the extension element to forcibly engage one another and frictionally resist relative rotation. With the extension secured to the coupling element, it is to be appreciated that a much longer portion of the system may be present outside the patient. Accordingly, a proximal portion of the extension may be secured or grasped by the user while the sheath is removed from the patient (414). When the sheath is fully removed, the proximal portion of the extension may remain exposed and secured by the user. In addition, the coupling element may also be exposed. The user may transition to controlling the system by grasping the coupling element, the cable tying element, the cable, or a distal portion of the extension element. The sheath may then be fully removed from the extension. A replacement sheath may then be advanced over a proximal end of the extension up to the coupling element, for example, and exposing the proximal end of the extension (416). The proximal end of the extension may be secured and the sheath may be advanced over the portion of the system remaining in the patient. The extension may then be removed (418).

The handle may be reattached to the system using steps that are generally the opposite of those previously described (420). For example, the core element may be placed over the coupling element, the proximal portion of the wire may be pressed through the slot and windows of the core element, the coupling element, and the cable tying element. The release element may be rotated the opposite direction as before to reengage the core element and the cable tying element, and then to disengage the coupling element and the cable tying element allowing the latter two elements freedom to move longitudinally. Further steps to remove the medical device or otherwise complete the medical procedure may also be performed (422). Upon proper placement of the medical device, the locking element may be advanced away from the control element and the control element may be actuated to release the engagement mechanism.

The above described system may also be used in situations where the delivery system has an insufficient length. That is, where a delivery system has insufficient length, steps the same or similar to the ones just mentioned may be used to remove the handle. An extension portion may be provided that is the same or similar to the one described above. Moreover, a handle with a longer proximal wire portion may be provided and the proximal portion of the extension may have features configured to engage the core element and the release element and transfer rotational forces from the handle to the coupling element. As such, the handle may be secured to the proximal end of the extension, the proximal portion of the wire may be threaded through the extension and may be secured into the coupling element as mentioned above. The release element may be rotated, causing the extension to rotate and causing the cable tying element and the core element to reengage via the extension element. Further rotation may also disengage the coupling element from the cable tying element.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A decoupling delivery system for delivering a medical device, the delivery system comprising:
a proximal end and a distal end;
a handle portion provided at the proximal end, the handle portion including a catheter engagement mechanism;
a device engagement mechanism provided at the distal end of the system for engaging the medical device; and
a catheter portion extending between the handle portion and the device engagement mechanism, wherein the catheter portion comprises:
a proximal catheter portion including a handle engagement mechanism;
a wire; and
a sheath, wherein the wire extends within the sheath;
wherein the handle portion is removably coupleable from the catheter portion by engaging and disengaging the catheter engagement mechanism with the handle engagement mechanism and wherein the medical device remains engaged by the device engagement mechanism when the handle portion is decoupled from the delivery system.

2. The delivery system of claim 1, wherein actuation of the handle portion advances or retracts the wire longitudinally within the sheath.

3. The delivery system of claim 1, wherein the device engagement mechanism is operable to engage and disengage the medical device in response to longitudinal motion of the wire.

4. The delivery of claim 1, wherein the device engagement mechanism comprises a pair of prongs that are connected to one another and biased in an open position.

5. The delivery system of claim 1, wherein decoupling of the handle portion from the catheter portion allows removal and replacement of the sheath while the system and medical device remain in a patient.

6. The delivery system of claim 1, wherein the handle further comprises a anti-release mechanism configured to prevent inadvertent release of the medical device.

7. The delivery system of claim 1, wherein the catheter engagement mechanism comprises a handle connector and a pusher wire cap at a distal end of the handle connector, wherein a push wire may be extended through the handle connector and into the pusher wire cap.

8. The delivery system of claim 1, wherein the proximal catheter portion further comprises a catheter shrink, a compression spring, a pull wire attachment, and a hypotube, wherein a pull wire may extend through the catheter shrink, hypotube, and pull wire attachment, and wherein when the handle portion is not coupled to the catheter portion, the compression spring pushes the pull wire attachment, keeping a distal end of the sheath closed and the medical device grasped.

9. The delivery system of claim 1, wherein the handle portion includes a proximal handle portion, a core, a distal handle portion, and a thumb ring.

10. The delivery system of claim 9, wherein the handle portion further comprises a biasing portion over the core, the biasing portion separating the proximal handle portion from the distal handle portion.

11. A decoupling delivery system for delivering a medical device, the delivery system comprising:
a proximal end and a distal end;
a handle portion provided at the proximal end, the handle portion including:
a core;
an engagement control; and
a catheter engagement mechanism;
a device engagement mechanism provided at the distal end of the system for engaging the medical device, the device engagement mechanism comprising a pair of prongs that are biased toward an open position; and
a catheter portion extending between the handle portion and the device engagement mechanism, wherein the catheter portion comprises:
a handle engagement mechanism;
a wire coupleable to the device engagement mechanism; and
a cable, wherein the wire extends within the cable;
wherein the handle portion is removably coupleable from the catheter portion by engaging and disengaging the catheter engagement mechanism with the handle engagement mechanism and wherein the medical device remains engaged by the device engagement mechanism when the handle portion is decoupled from the delivery system;
wherein actuation of the handle portion advances or retracts the wire longitudinally within the cable and wherein such actuation operates to open and close the device engagement mechanism.

12. The delivery system of claim 11, wherein the catheter engagement mechanism and the handle engagement mechanism have complementary configurations for engagement.

13. The delivery system of claim 11, wherein the core is configured for receiving a distal portion the cable and wire of the catheter portion.

14. The delivery system of claim 11, wherein the engagement control is biased such that the engagement mechanism is biased in a clamping position.

15. The delivery system of claim 14, wherein the engagement control is moved to move the engagement mechanism from the clamping position.

16. The delivery system of claim 11, wherein the handle further comprises a distal handle portion arranged on the core and configured to prevent inadvertent advancement of the engagement control.

17. The delivery system of claim 11, wherein the handle had a locked position and an unlocked position.

18. A method of delivering a medical device to a position in a body of a patient, the method comprising:
providing a decoupling delivery device comprising:
a proximal end and a distal end;
a handle portion provided at the proximal end, the handle portion including a catheter engagement mechanism;
a device engagement mechanism provided at the distal end of the system for engaging the medical device; and
a catheter portion extending between the handle portion and the device engagement mechanism, wherein the catheter portion comprises:
a proximal catheter portion including a handle engagement mechanism;
a wire; and
a sheath, the wire extending within the sheath;
wherein the handle portion is coupled and decoupled from the delivery system by engaging and disengaging the catheter engagement mechanism with the handle engagement mechanism and wherein the medical device remains engaged by the device engagement mechanism when the handle portion is decoupled from the delivery system;
wherein actuation of the handle portion advances or retracts the wire longitudinally within the sheath;
using the handle portion to actuate the device engagement mechanism and engage the medical device to the distal end of the decoupling delivery device;
inserting the medical device and at least a portion of the delivery system into the patient;
advancing the medical device to a delivery location;
advancing the medical device out of the distal end of the sheath;
decoupling the handle portion from the system;
recoupling the handle portion to the system;
using the handle portion to actuate the device engagement mechanism to release the medical device; and
withdrawing the medical delivery system.

19. The method of claim 18, further comprising replacing the sheath after decoupling the handle portion from the system.

20. The method of claim 18, further comprising adding an extension after decoupling the handle from the system.
